(19) United States
(12) Patent Application Publication
(10) Pub. No.: US 2014/0222096 A1
(43) Pub. Date: Aug. 7, 2014

(54) CELL PHONE WITH AUTOMATIC EXTERNAL DEFIBRILLATOR

(71) Applicant: NATIONAL CENTRAL UNIVERSITY, Taoyuan County (TW)

(72) Inventors: Yu-Feng HU, Taipei City (TW); Bo-Jau KUO, Taipei (TW); Shih-Ann CHEN, Taipei City (TW); Men-Tzung LO, Taoyuan County (TW); Norden-Eh HUANG, Taoyuan County (TW)

(73) Assignee: NATIONAL CENTRAL UNIVERSITY, Taoyuan County (TW)

(21) Appl. No.: 13/960,544

(22) Filed: Aug. 6, 2013

(30) Foreign Application Priority Data
Feb. 7, 2013 (TW) 102104761

(51) Int. Cl. A61N 1/39

(52) U.S. Cl. CPC A61N 1/3968
USPC 607/5

(57) ABSTRACT

The present invention provides a cell phone and a case, wherein the case includes a external system and a internal system, wherein the external system includes a signal receive part, a cell phone case, a connector and a defibrillator part, wherein the internal portion includes an power supply part, a amplifying part and a signal processing part; the present invention also provides a method for removal of cardiac ventricular fibrillation.

### CROSS-REFERENCE TO RELATED APPLICATIONS

This Non-provisional application claims priority under 35 U.S.C. §119(a) on Patent Application No(s). 102104761 filed in Taiwan, Republic of China, 02, 07, 2013, the entire contents of which are hereby incorporated by reference.

### FIELD OF THE INVENTION

The field of the invention is related to a cell phone with automatic external defibrillator and the method of using thereof.

### BACKGROUND OF THE INVENTION

In the 1970s, the development of Automatic external defibrillator (AED) had saved many precious lives of patients with heart disease.

The general automated external defibrillator includes two kinds of features, The first one feature of the ECG interpretation is, the automatic external defibrillator with ECG analysis software would automatically interpretive of the patient's electrocardiogram ,and confirm whether to operate an electric shock when the patients appear ventricular frequency pulse (ventricular tachycardia), ventricular fibrillation (ventricular fibrillation).The software would inform the operator whether to recommend defibrillation through the voice system, in comparison to the diagnosis of the clinicians, the automated external defibrillator ECG analysis software can be more sensitive and specific than 95%. The other feature of this automatic defibrillator is, the software analysis would confirm with suitable shock condition, make the operator press the button to give an electric shock and defibrillate through the voice instruction, and would also accurately control the energy without causing injury to the patient.

Generally, AED equipment often conduction flow through two electrode patches affixed to the patient, and the conventional external defibrillation equipment is bulky and is not easy for carry, which can be only placed at specific places. In Japan, there is only one set of defibrillator device in an average of about one square kilometers, and the utilization rate is low of only 3.6%. Besides, the density and utilization rate is even lower in Taiwan than in advanced countries, such as Europe and the United States. Under the sudden cardiac disorders of patients, it is difficult to set designated AED equipment properly, so there are also many patients died due to sudden cardiac disorder. In addition, the price of existing automated external defibrillator unit is too high, the general public cannot afford is also one of a problem. An AED costs about 10 to 20 million NTD and requires regular maintenance, so it is hard to popularization.

In order to solve above problems, lots of R&D units have developed related products, such as Taiwan Patent Number 200950844 that disclose an automatic external defibrillator, which is related to a structure with content of utilize of automated external defibrillator by a built-in or plus dedicated automatically informed control module, and with providing a level stacking capability expansion on the automatically informed control module, so as to provide an automatic paging communication function of real-time status transmission, public mobile networks and area wireless network audiovisual/data communications, as well as outdoor and indoor positioning, and abnormal normal or error status of the automatic external defibrillator.

Additionally, the U.S. patent No. S20110046688A1 also provides a notebook, tablet PC, PDA or other communication device with automated external defibrillation device (AED) combined system, and provides an modified notebook, tablet PC, PDA able or other communication device which can be applied with such as method of automated external defibrillators, so that users can take advantage of the defibrillators of the notebook, tablet PC, a desktop computer or other communications device to treat cardiac arrest victims. The technology can be applied on each notebook, tablet computer or communications device, which is therefore considered as the automated external defibrillator (AED).

These two patents disclose the integration of automated external defibrillators in the use of communication devices, but it is not combined an automated external defibrillator with a cell phone cases for more application.

### SUMMARY OF THE PRESENT INVENTION

The present invention provides an automatic external defibrillator device, which is installed on the phone. It utilize the popularity of mobile phones to increase the popularizing rate of the defibrillator, so as to reduce delay of rescue time and the inconvenience, and the overall survival rate and prognosis of patients would be increased improved. In addition, the additional automatic external defibrillator function on the case and software of the cell phone, to utilize the original components and functions of the cell phone, and the phone battery is also as a power supply, and the software is support by phone. This make the components small and the significantly reduce the price.

Accordingly, the present invention provides a cell phone and a case, wherein the case includes an external system and an internal system, wherein the external system is a combination in the cell phone's external, and the internal system is an operating mode of the cell phone, wherein the external system includes: a signal receive patch set, which can be attached on a chest of a patient and collect a cardiac ventricular fibrillation signal; and an electric shock patch set, which can emit a electric shock flow transmit to the chest of the patient; the internal system includes: a power supply part; a signal processing part, which is connected with the signal receive patch set and can transform the ventricular fibrillation signal to a digital signal; a amplifying part, which is connected with the electric shock patch set and can amplify the electric shock flow from the electric shock patch set; and a connector part, which is connected with the electric shock patch set and the cell phone.

Preferably, the signal receive patch set is composed of a pair of patches which attach on chest, both ends of the patient's heart side.

Preferably, the ventricular fibrillation signal is an ECG signal.

Preferably, the cell phone includes a signal analysis software.

Preferably, the signal analysis software can analyze the digital signal; if the analyzed digital signal is abnormal from normal patient, then drive the power supply part.

Preferably, the signal analysis software can automatically dial to an emergency and care center to further provide the location of the cell phone.

Preferably, the electric shock patch set is composed of a pair of patches which attach on chest, both ends of the patient's heart side.

Preferably, the patch of the electric shock patch set provide a bidirectional electric shock, or/and a unidirectional electric shock.

Preferably, the bidirectional electric shock flow of the electric shock patch set is 200 Joule.

Preferably, the unidirectional electric shock flow of the electric shock patch set is 360 Joule.

Preferably, the power supply part can provide a charging flow to the electric shock patch set, or/and provide the charging flow to the cell phone.

Preferably, the patch of the electric shock patch set is a bidirectional electric shock, or/and a unidirectional electric shock.

Preferably, the electric shock patch set is connected with the signal receive patch set as an electric shock-signal receive patch set.

In summary, the present invention provides a cell phone with automatic external defibrillator, which can detect the ECG signal of a patient and analyze it, then determine whether to drive a electric shock and drive the automatically dial to an emergency and care center to further provide the location of the cell phone, so as to shorten the rescue time.

### BRIEF DESCRIPTION OF THE DRAWINGS

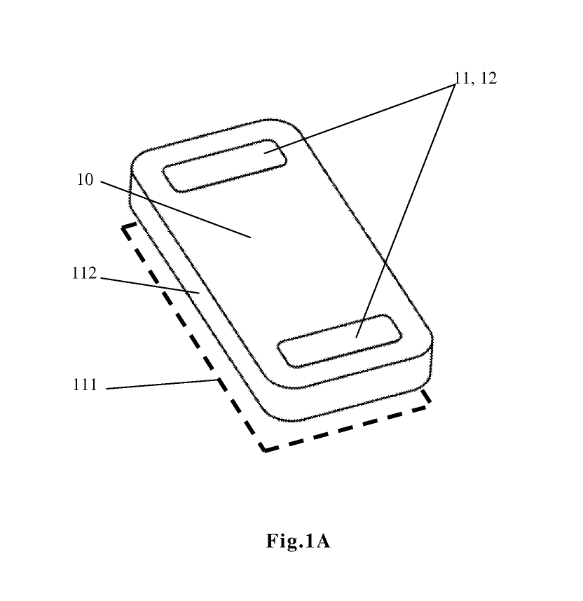

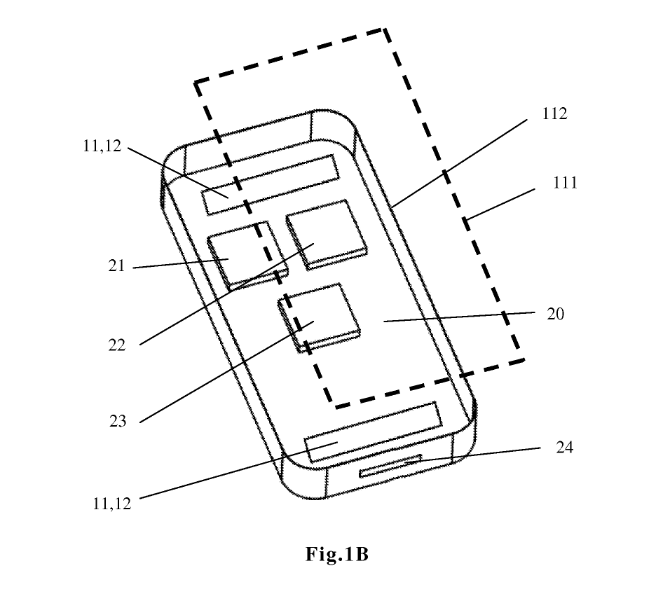

The FIG. 1A is the schematic diagram of the external system.

The FIG. 1B is the schematic diagram of the internal system.

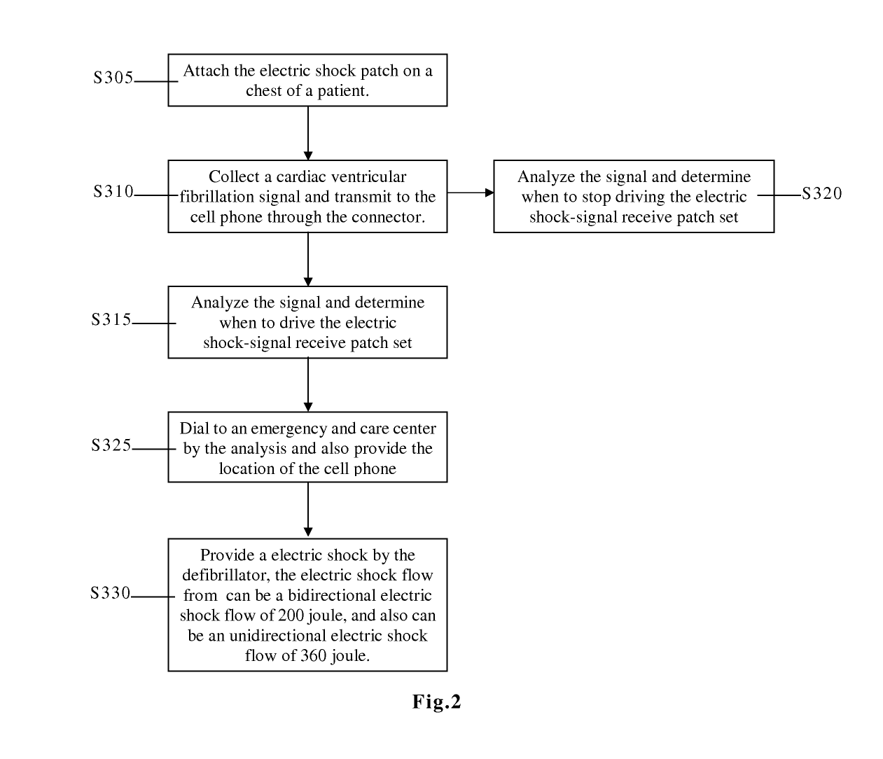

The FIG. 2 is the flowchart of automatically defibrillation method of cell phone with automatic external defibrillator of the present invention.

Symptom of the Drawings

| | | | |
|---|---|---|---|
| 10 | external system | 11 | signal receive patch set |
| 111 | Cell phone | 112 | case |
| 12 | electric shock patch set | 20 | internal system |
| 21 | power supply part | 22 | signal processing part |
| 23 | amplifying part | 24 | connector part |
| Steps | S305~S330 | | |

### DETAILED DESCRIPTION

The present invention provides a cell phone with automatic external defibrillator, which can refer to FIG. 1A and FIG. 1B, includes a cell phone 111 and a case 112, the case 112 includes an external system 10 and an internal system 20, wherein the external system 10 is located on external portion of the case 112, and the internal system 20 is located on internal portion of the case 112, wherein the external system 10 includes: a signal receive patch set 11, which can be attached on a chest of a patient and collect a cardiac ventricular fibrillation signal; and an electric shock patch set 12, which can emit a electric shock flow transmit to the chest of the subject; the internal system 20 includes: an power supply part 21; a signal processing part 22, which is connected with the signal receive patch set 11 and can transform the ventricular fibrillation signal to a digital signal; a amplifying part 23, which is connected with the electric shock patch set 12 and can amplify the electric shock flow from the electric shock patch set 12; and a connector part 24, which is connected with the electric shock patch set 12 and the cell phone 111.

The better embodiment can refer to FIG. 2, which can clarify the external defibrillator method of the present invention, includes installing the automatic external defibrillator on a cell phone. In the embodiment, the cell phone case is installed on the cell phone by a general case installing way; then the electric shock patch set of the cell phone with external defibrillator is attached on a chest of a subject, and the signal receive patch set is attached on the chest of the patient (Step S305) to collect a cardiac ventricular fibrillation signal from the patient. In the embodiment, the signal receive patch is a signal receive patch set, and the electric shock patch set is connected with the signal receive patch as an electric shock-signal receive patch set, which is attached on a chest of a patient to collect a cardiac ventricular fibrillation signal. The collected cardiac ventricular fibrillation signal is transformed to a digital signal and is transmitted to the cell phone through the connector. An signal analysis software is installed in the cell phone, which can further analyze the digital signal and determine when to drive the electric shock-signal receive patch set (Step S315), and also determine when to stop driving the electric shock-signal receive patch set by received digital signal (Step S320). In addition, it can determine whether to automatically dial to an emergency and care center by the analysis. In the embodiment, the emergency and care center is 119 Fire Department, and also provide the location of the cell phone, so as to shorten the rescue time. When the shock-signal receive patch set is drive, the power supply part can provide a charging flow to the shock-signal receive patch set. When the shock-signal receive patch set is drive, the amplifying part can amplify the electric shock flow from the shock-signal receive patch set. In the embodiment, the electric shock flow can be a bidirectional electric shock flow of 200 joule, and also can be an unidirectional electric shock flow of 360 joule (Step S330). When the subject received the electric shock, the signal processing part can further analyze the ventricular fibrillation signal and determine whether to stop driving the electric shock-signal receive patch set. When the electric shock-signal receive patch set is stop driving, the power of the phone can transmit the charging flow to the power supply part. Besides, the power supply part also can be a backup power supply of the cell phone.

In summary, the cell phone with automatic external defibrillator of the present invention can be portable by accessorized on a cell phone, which is light, simple and with high popularizing rate. This provides a quick and safe first aid when the patient with heart disease is under emergency. In addition, the present invention can utilize the original function of cell phone to dial to the emergency and care center, and transmit the location of the patient to shorten the transportation time to the hospital, and rescue the patient timely.

What is claimed is:

1. A defibrillator which can be an component of a cell phone and a case ,wherein the case includes an external system and an internal system, wherein the external system is a combination in the cell phone's external, and the internal system is an internal operating mode of the cell phone, wherein
the external system includes:
a signal receive patch set, which can be attached on a chest of a patient and collect a cardiac ventricular fibrillation signal; and
an electric shock patch set, which can emit a electric shock flow transmit to the chest of the patient;
the internal system includes:
a power supply part;
a signal processing part, which is connected with the signal receive patch set and can transform the ventricular fibrillation signal to a digital signal;
an amplifying part, which is connected with the electric shock patch set and can amplify the electric shock flow from the electric shock patch set; and
a connector part, which is connected with the electric shock patch set and the cell phone.

2. The defibrillator in claim 1, wherein the signal receive patch set is composed of a pair of patches.

3. The defibrillator in claim 1, wherein the ventricular fibrillation signal is an ECG signal.

4. The defibrillator in claim 1, wherein the cell phone includes a signal analysis software.

5. The defibrillator in claim 4, wherein the signal analysis software can analyze the digital signal;
if the analyzed digital signal is abnormal from normal patient, then drive the power supply part.

6. The defibrillator in claim 4, wherein the signal analysis software can automatically dial to an emergency and care center to further provide the location of the cell phone.

7. The defibrillator in claim 1, wherein the electric shock patch set is composed of a pair of patches.

8. The defibrillator in claim 1, wherein the electric shock patch set provide a bidirectional electric shock, or/and a unidirectional electric shock.

9. The defibrillator in claim 8, wherein the bidirectional electric shock flow of the electric shock patch set is 200 Joule.

10. The defibrillator in claim 8, wherein the unidirectional electric shock flow of the electric shock patch set is 360 Joule.

11. The defibrillator in claim 1, wherein the power supply part can provide a charging flow to the electric shock patch set, or/and provide the charging flow to the cell phone.

12. The defibrillator in claim 1, wherein the electric shock patch set is connected with the signal receive patch set as an electric shock-signal receive patch set.
